(19) United States
(12) Patent Application Publication
(10) Pub. No.: US 2024/0000589 A1
(43) Pub. Date: Jan. 4, 2024

(54) STENT

(71) Applicant: Acandis GmbH, Pforzheim (DE)

(72) Inventors: Andreas Ding, Karlsruhe (DE); Andrés Braschkat, Bretten (DE); Andreas Schüssler, Pfinztal (DE)

(21) Appl. No.: 18/037,737

(22) PCT Filed: Nov. 16, 2021
(86) PCT No.: PCT/EP2021/081786

§ 371 Date: May 18, 2023

(30) Foreign Application Priority Data
Nov. 26, 2020 (DE) 20 2020 106 808.3
Jul. 2, 2021 (DE) 10 2021 117 096.6

(51) Int. Cl. A61F 2/90

(52) U.S. Cl. CPC A61F 2/90

(57) ABSTRACT

Stent including a mesh of wires having a radiopaque core material and a superelastic shell material and defining loops at a first stent end and open wire ends at a second stent end. The number of wire ends is double the number of loops; and more than 10 vol. %, in particular more than 20 vol. %, more particularly at least 25 vol. %, in particular at least 30 vol. % of each wire is constituted by the core material. The mesh includes (i) 48 wires, each having a wire diameter of 0.038 mm and an outer diameter of 3.65 mm or 4.15 mm or 4.65 mm; (ii) 52 wires each having a wire diameter of 0.042 mm and an outer diameter of 5.17 mm or 5.67 mm or 6.17 mm; or (iii) 64 wires each having a wire diameter of 0.046 mm or 0.05 mm and an outer diameter of 7.18 mm or 8.20 mm.

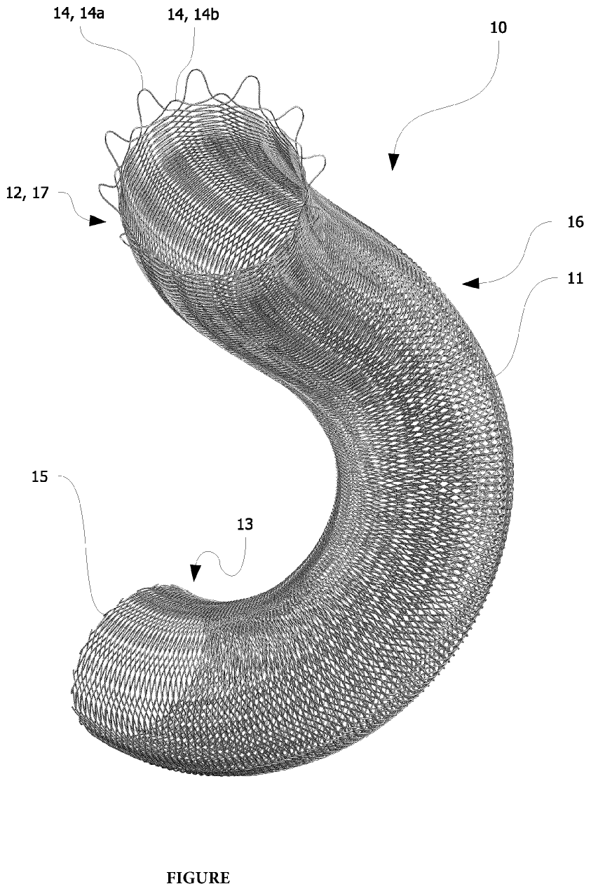

The invention relates to a stent with a meshwork of wires. Stents of this type are known in the art. Providing at least some of the wires with a radiopaque core material and a superelastic sheath material so that the stent or the meshwork can be seen under radiographic monitoring is also known. Furthermore, stents are also known which have loops at least at one end of the stent and which therefore act atraumatically, i.e. the danger of injury upon implantation of the stent is reduced.

Currently known stents usually comprise wires with a proportion of core material of at most 20% by volume. These known stents are indeed visible under radiographic monitoring. However, the detectability of stents under radiographic monitoring needs to be improved.

DE 10 2015 107 291 A1, for example, describes a stent with wires which have a radiopaque core wire, wherein the cross sectional area of the core wire has a proportion of at most 20% with respect to the total cross sectional area of the wire.

DE 10 2018 133 345 A1 discloses a stent which is braided from a single wire which has a radiopaque core material with a proportion of core material of 27%. The known stent is characterized by a particular radial force due to its single wire construction, but this is not 25 desirable for every intended application.

A stent is known from DE 10 2019 104 827 A1 which, because of a particular surface treatment, has a particularly low tendency to release nickel and therefore provides improved biocompatibility.

DE 10 2019 104 828 A1 concerns a stent which is formed from wires which have a radiopaque core material. By means of a pre-specified relationship between the cross sectional area of the core material with respect to the total cross sectional area of the wire, good visibility of this stent under radiographic monitoring is obtained. The present invention is a further development of that stent in order to gain access to other fields of application.

Thus, the objective of the invention is to provide a stent with a meshwork of wires which have an improved radiopacity.

In accordance with the invention, this objective is achieved by means of the subject matter of claim 1.

Thus, the invention is based on the idea of providing a stent with a meshwork of wires, which respectively have a radiopaque core material and a superelastic sheath material. The wires form loops at a first stent end and open wire ends at a second stent end. The number of wire ends is twice as high as the number of loops. In accordance with the invention, more than 10% by volume, in particular more than 20% by volume, in particular at least 25% by volume, in particular at least 30% by volume of each wire is formed by the core material.

Increasing the proportion of core material significantly increases the radiopacity of the stent.

A further improvement in the radiopacity is obtained by raising the number of wires and the wire diameter. Thus, in accordance with the invention, the meshwork has 48 wires each with a wire diameter of 0.038 mm. In this variation, the outside diameter of the meshwork is preferably 3.65 mm or 4.15 mm or 4.65 mm.

In a further variation of the stent in accordance with the invention, the meshwork has 52 wires each with a wire diameter of 0.042 mm. In this variation, the outside diameter of the meshwork is preferably 5.17 mm or 5.67 mm or 6.17 mm.

As an alternative, the stent comprises a meshwork with 64 wires, wherein the wires each have a wire diameter of 0.046 mm or 0.05 mm. In this variation, the outside diameter of the meshwork is preferably 7.18 mm or 8.20 mm. In accordance with the invention, a meshwork with an outside diameter of 7.18 mm has 64 wires, which each have a wire diameter of 0.046 mm. In contrast, a meshwork with an outside diameter of 8.20 mm comprises 64 wires with a wire diameter of 0.05 mm.

All of the aforementioned values for the outside diameters have a tolerance of ±0.2 mm. The aforementioned wire diameter has a tolerance of ±0.003 mm. Furthermore, in all of the embodiments, all of the wires of the meshwork or stent have a core material and a sheath material.

In a further variation of the invention, the meshwork has a cylindrical section and a conical section. The conical section may form the first stent end, in particular exclusively. Preferably, the conical section has an angle of twist, which is different from that of the cylindrical section. The “angle of twist” designates the angle of the respective wires with respect to the longitudinal axis of the stent. Preferably, wires which are twisted in different spiral directions and which cross over or under each other have identical angles of twist.

Thus, in a preferred variation, a first angle of twist in the cylindrical section is 75° and/or a second angle of twist in the conical section is 60°. A transitional section, which has a smaller, preferably third angle of twist and/or a higher porosity than the cylindrical section may be provided between the cylindrical section and the conical section. In this regard, the first angle of twist preferably has a tolerance of ±2° and/or the second angle of twist has a tolerance of ±4°. The angle of twist, in particular the third angle of twist, in the transitional section may have a tolerance of ±3°.

Preferably, the length of the meshwork is between 12 mm and 38 mm. Specifically, the meshwork may have one of the following lengths: 12 mm, 13 mm, 15 mm, 16 mm, 19 mm, 20 mm, 22 mm, 23 mm, 24 mm, 28 mm, 30 mm, 31 mm, 37 mm, 38 mm.

The first stent end may have a length of 4 mm. The “first stent end” designates the region in which the loops are formed. In this regard, the distance from the vertex of the loop to the first crossover point of two wires is measured in order to determine the length. This region is designated the first stent end and preferably has a length of 4 mm.

All of the values given for the lengths may have a tolerance of ±1 mm.

In order to be able to deliver it via a catheter as well as have good biocompatibility, the meshwork, in particular the wires, preferably has a shimmering blue oxide surface. The oxide surface facilitates delivery through a catheter and improves biocompatibility. The shimmering blue visual appearance aids users of the stent in differentiating it from other stents.

Preferably, the oxide surface comprises or consists of a mixed oxide layer. The mixed oxide layer may comprise titanium oxynitride. Preferably, the layer thickness of the mixed oxide layer is between 150 nm and 400 nm, in particular between 200 nm and 350 nm, in particular between 250 nm and 300 nm.

Because using wires with a radiopaque core material improves the radiopacity, in particularly preferred embodiments, the meshwork does not have any additional radiographic markers. The meshwork may exclusively consist of the wires. In this manner, no additional elements are present which could protrude beyond the wire diameter and could result in making the delivery capability or compressibility of the meshwork more difficult. In other words, the meshwork or stent is easily compressible well and can be introduced into a blood vessel through a catheter. Even in the implanted state, the risk of irritating tissue, which arises because elements such as additional radiographic markers, which protrude beyond the stent surface can penetrate into the tissue, is averted.

In a preferred variation, the stent consists of the meshwork. In other words, the stent preferably does not have any components other than the meshwork.

In contrast, in an alternative embodiment of the stent, the stent has the meshwork and at least one, preferably three radiographic markers. The radiographic marker or the radiographic markers are preferably disposed in the loops at the first stent end. In particular, the radiographic marker or the radiographic markers may be formed by a sleeve, which is crimped onto the wire at the loops. Advantageously, the stent in this variation has only the meshwork and one or more radiographic markers, preferably at the loops.

In addition, in all of the embodiments, the core material of the wire is preferably platinum. A platinum alloy may also be envisaged as the core material, in particular a platinum-iridium alloy. The sheath material is preferably a nickel-titanium alloy. The nickel-titanium alloy has shape memory or superelastic properties. This makes the stent self-expandable.

The invention will now be described in more detail with the aid of an exemplary embodiment and with reference to the accompanying diagrammatic drawing. The single FIGURE shows a perspective view of a stent in accordance with the invention.

The stent consists of a meshwork 10, which is formed from a plurality of wires 11. The meshwork has a first stent end 12 and a second stent end 13. Loops are formed at the first stent end 12. Thus, the wires 11 turn back on themselves at the first stent end 12 so that the loops 14 are formed. It should in particular be noted that every second loop 14 is set back with respect to the adjacent loop 14. Thus, protruding loops 14a and set back loops 14b are provided. The staggered arrangement of the loops 14a, 14b facilitates the compressibility of the first stent end 12.

The first stent end 12 preferably widens conically. The vertices of the loops 14, in particular the protruding loops 14a, are therefore orientated outwardly. This configuration improves anchoring of the first stent end 12 in the implanted state.

Open wire ends 15 can be seen at the second stent end 13 of the meshwork 10. Thus, the wires 11 end at the second stent end 13.

The mesh structure comprises a total of 48, 52 or 64 wires 11, which respectively form 24 or 26 or 32 loops 14. Here, 12 or 13 or 16 loops 14 are configured as protruding loops 14a or as set back loops 14b.

Preferably, the stent is offered in a variety of dimensions. An overview of the preferred embodiments of the stent is shown in the table below.

| | | Number | Wire diameter | Angle of | | | Length | cylindrical |
|---|---|---|---|---|---|---|---|---|
| | Stent | of loops | d/mm | twist | | Total | of loop | Length of |
| | diameter | (wires) | (tolerance | AT- | AT- | length | segment | section |
| Embodiment | OD stent/ | nl | according to | L3/° | L2/° | L1/mm | L2/mm | L3/mm |
| number | mm (±0.2 mm) | (nw)/— | specifications) | (±2°) | (±4°) | (±1 mm) | (±1 mm) | (±1 mm) |
| 1 | 3.65 | 24 (48) | 0.038 | 75 | 60 | 22 | 4 | 19 |
| 2 | 3.65 | 24 (48) | 0.038 | 75 | 60 | 28 | 4 | 25 |
| 3 | 4.15 | 24 (48) | 0.038 | 75 | 60 | 12 | 4 | 8 |
| 4 | 4.15 | 24 (48) | 0.038 | 75 | 60 | 15 | 4 | 11 |
| 5 | 4.15 | 24 (48) | 0.038 | 75 | 60 | 19 | 4 | 15 |
| 6 | 4.15 | 24 (48) | 0.038 | 75 | 60 | 22 | 4 | 18 |
| 7 | 4.15 | 24 (48) | 0.038 | 75 | 60 | 29 | 4 | 25 |
| 8 | 4.65 | 24 (48) | 0.038 | 75 | 60 | 12 | 4 | 8 |
| 9 | 4.65 | 24 (48) | 0.038 | 75 | 60 | 15 | 4 | 11 |
| 10 | 4.65 | 24 (48) | 0.038 | 75 | 60 | 19 | 4 | 15 |
| 11 | 4.65 | 24 (48) | 0.038 | 75 | 60 | 22 | 4 | 18 |
| 12 | 4.65 | 24 (48) | 0.038 | 75 | 60 | 29 | 4 | 25 |
| 13 | 5.17 | 26 (52) | 0.042 | 75 | 60 | 12 | 4 | 8 |
| 14 | 5.17 | 26 (52) | 0.042 | 75 | 60 | 15 | 4 | 11 |
| 15 | 5.17 | 26 (52) | 0.042 | 75 | 60 | 19 | 4 | 15 |
| 16 | 5.17 | 26 (52) | 0.042 | 75 | 60 | 23 | 4 | 19 |
| 17 | 5.17 | 26 (52) | 0.042 | 75 | 60 | 30 | 4 | 26 |
| 18 | 5.17 | 26 (52) | 0.042 | 75 | 60 | 37 | 4 | 33 |
| 19 | 5.67 | 26 (52) | 0.042 | 75 | 60 | 13 | 4 | 9 |
| 20 | 5.67 | 26 (52) | 0.042 | 75 | 60 | 16 | 4 | 12 |
| 21 | 5.67 | 26 (52) | 0.042 | 75 | 60 | 20 | 4 | 16 |
| 22 | 5.67 | 26 (52) | 0.042 | 75 | 60 | 24 | 4 | 20 |
| 23 | 5.67 | 26 (52) | 0.042 | 75 | 60 | 31 | 4 | 27 |
| 24 | 5.67 | 26 (52) | 0.042 | 75 | 60 | 38 | 4 | 34 |
| 25 | 6.17 | 26 (52) | 0.042 | 75 | 60 | 13 | 4 | 9 |
| 26 | 6.17 | 26 (52) | 0.042 | 75 | 60 | 16 | 4 | 12 |
| 27 | 6.17 | 26 (52) | 0.042 | 75 | 60 | 20 | 4 | 16 |
| 28 | 6.17 | 26 (52) | 0.042 | 75 | 60 | 24 | 4 | 20 |
| 29 | 6.17 | 26 (52) | 0.042 | 75 | 60 | 31 | 4 | 27 |
| 30 | 6.17 | 26 (52) | 0.042 | 75 | 60 | 38 | 4 | 34 |
| 31 | 7.18 | 32 (64) | 0.046 | 75 | 60 | 16 | 4 | 12 |
| 32 | 7.18 | 32 (64) | 0.046 | 75 | 60 | 20 | 4 | 16 |
| 33 | 7.18 | 32 (64) | 0.046 | 75 | 60 | 24 | 4 | 20 |
| 34 | 7.18 | 32 (64) | 0.046 | 75 | 60 | 31 | 4 | 27 |
| 35 | 7.18 | 32 (64) | 0.046 | 75 | 60 | 38 | 4 | 34 |
| 36 | 8.20 | 32 (64) | 0.05 | 75 | 60 | 16 | 4 | 12 |
| 37 | 8.20 | 32 (64) | 0.05 | 75 | 60 | 20 | 4 | 16 |
| 38 | 8.20 | 32 (64) | 0.05 | 75 | 60 | 24 | 4 | 20 |
| 39 | 8.20 | 32 (64) | 0.05 | 75 | 60 | 31 | 4 | 27 |
| 40 | 8.20 | 32 (64) | 0.05 | 75 | 60 | 38 | 4 | 34 |

### LIST OF REFERENCE NUMERALS

10 meshwork
11 wire
12 first stent end
13 second stent end
14 loop
14a protruding loop
14b set back loop
15 open wire end
16 cylindrical section
17 conical section

1-14. (canceled)

15. A stent comprising:
a meshwork of wires having a radiopaque core material and a superelastic sheath material, the wires configured and arranged to form loops at a first stent end and open wire ends at a second stent end, wherein a number of the open wire ends is twice the number of the loops, and more than 10% by volume of each wire is formed by the radiopaque core material, and
wherein the meshwork has 48 wires each with a wire diameter of 0.038 mm, and
wherein the meshwork has an outside diameter of one of 3.65 mm, 4.15 mm, or 4.65 mm.

16. The stent according to claim 15, wherein the outside diameter has a tolerance of ±0.2 mm.

17. The stent according to claim 15, wherein the meshwork has a cylindrical section and a conical section, and wherein the conical section forms the first stent end and has an angle of twist different from that of the cylindrical section.

18. The stent according to claim 17, wherein a first angle of twist in the cylindrical section is 75° and a second angle of twist in the conical section is 60°.

19. The stent according to claim 18, wherein the first angle of twist has a tolerance of ±2° and the second angle of twist has a tolerance of ±4°.

20. The stent according to claim 17, wherein a transitional section having a smaller angle of twist and a higher porosity than the cylindrical section is provided between the cylindrical section and the conical section.

21. The stent according to claim 15, wherein a length of the meshwork is between 12 mm and 38 mm.

22. The stent according to claim 21, wherein the length has a tolerance of ±1 mm.

23. The stent according to claim 15, wherein a length of the meshwork is one of 12 mm, 13 mm, 15 mm, 16 mm, 19 mm, 20 mm, 22 mm, 23 mm, 24 mm, 28 mm, 30 mm, 31 mm, 37 mm, or 38 mm.

24. The stent according to claim 23, wherein the length has a tolerance of ±1 mm.

25. The stent according to claim 15, wherein the first stent end has a length of 4 mm.

26. The stent according to claim 25, wherein the length has a tolerance of ±1 mm.

27. The stent according to claim 15, wherein the meshwork has a shimmering blue oxide surface.

28. The stent according to claim 27, wherein the oxide surface is formed by a mixed oxide layer of titanium oxynitride and having a layer thickness of between 150 nm and 400 nm.

29. The stent according to claim 15, wherein the meshwork has no additional radiographic markers and consists exclusively of the wires.

30. A stent comprising:
a meshwork of wires having a radiopaque core material and a superelastic sheath material, the wires configured and arranged to form loops at a first stent end and open wire ends at a second stent end, wherein a number of the open wire ends is twice the number of the loops, and more than 10% by volume of each wire is formed by the radiopaque core material, and
wherein the meshwork has 52 wires each with a wire diameter of 0.042 mm and having a tolerance of ±0.2 mm, and
wherein the meshwork has an outside diameter of one of 5.17 mm, 5.67 mm, or 6.17 mm.

31. The stent according to claim 30, wherein the meshwork has a cylindrical section and a conical section, and wherein the conical section forms the first stent end and has an angle of twist different from that of the cylindrical section.

32. The stent according to claim 31, wherein a transitional section having a smaller angle of twist and a higher porosity than the cylindrical section is provided between the cylindrical section and the conical section.

33. A stent comprising:
a meshwork of wires having a radiopaque core material and a superelastic sheath material, the wires configured and arranged to form loops at a first stent end and open wire ends at a second stent end, wherein a number of the open wire ends is twice the number of the loops, and more than 10% by volume of each wire is formed by the radiopaque core material, and
wherein the meshwork has 64 wires each with a wire diameter of one of 0.046 mm or 0.05 mm, and
wherein the meshwork has an outside diameter of one of 7.18 mm or 8.20 mm.

34. The stent according to claim 33, wherein the meshwork has a cylindrical section and a conical section, and wherein the conical section forms the first stent end and has an angle of twist different from that of the cylindrical section.
